SEQUENCE LISTING (7 sequences)

SEQ ID NO: 1 - DNA, 10 bp, artificial sequence
  oligonucleotide with consensus binding site for TP53
rrrcwwgyyy

SEQ ID NO: 2 - DNA, 20 bp, artificial sequence
  primer_binding: TP53 Exon 2-4 forward primer
cagccattcttttcctgctc

SEQ ID NO: 3 - DNA, 20 bp, artificial sequence
  primer_binding: TP53 Exon 2-4 reverse primer
agggtgtgatgggatggata

SEQ ID NO: 4 - DNA, 20 bp, artificial sequence
  primer_binding: TP53 Exon 5-6 forward primer
gtttctttgctgccgtcttc

SEQ ID NO: 5 - DNA, 20 bp, artificial sequence
  primer_binding: TP53 Exon 5-6 reverse primer
ttgcacatctcatggggtta

SEQ ID NO: 6 - DNA, 20 bp, artificial sequence
  primer_binding: TP53 Exon 7-9 forward primer
ggaggctgaggaaggagaat

SEQ ID NO: 7 - DNA, 20 bp, artificial sequence
  primer_binding: TP53 Exon 7-9 reverse primer
ttgaaagctggtctggtcct

(19) United States
(12) Patent Application Publication
(10) Pub. No.: US 2008/0234138 A1
(43) Pub. Date: Sep. 25, 2008

(54) TP53 gene expression and uses thereof

(76) Inventors: John D. Shaughnessy, Roland, AR (US); Bart Barlogie, Little Rock, AR (US)

Correspondence Address: Benjamin Aaron Adler;ADLER & ASSOCIATES, 8011 Candle Lane, Houston, TX, 77071, US

(21) Appl. No.: 11/999,766

(22) Filed: Dec. 7, 2007

Related U.S. Application Data
(60) Provisional application No. 60/873,840, filed on Dec. 8, 2006.

(51) Int. Cl. C40B 30/04; C12Q 1/68

(52) U.S. Cl. 506/9; 435/6

(57) ABSTRACT

The present invention is drawn to diagnosis, prognosis and treatment of multiple myeloma. In this regard, the present invention discloses importance of down-regulation of TP3 gene in multiple myeloma and its use as an independent progostic indicator of multiple myeloma. Additionally, the present invention also discloses novel-TP53 associated genes and demonstrates the clinical relevance of these alterations to disease progression.

### CROSS REFERENCE TO RELATED APPLICATION

This non-provisional application claims benefit of provisional application U.S. Ser. No. 60/873,840 filed on Dec. 8, 2006, now abandoned.

### FEDERAL FUNDING LEGEND

This invention was supported in part by National Institutes of Health, Campus Account No: CA55819. Consequently, the federal government has certain rights in this invention.

### BACKGROUND OF THE INVENTION

1. Field of the Invention

The present invention relates generally to the field of cancer research. More specifically, the present invention relates to correlating TP53 gene status with disease progression and outcome of a large, uniformly-treated population of patients with myeloma.

2. Description of the Related Art

The genetic lesions important in the pathogenesis and prognosis of multiple myeloma continue to be elucidated. Gene expression profiles can be used to identify high-risk diseases [1]. However, one of the surprising findings of this study was that variation in TP53 gene expression was not indicative of high-risk disease.

Using high-resolution array comparative genomic hybridization (aCGH), 87 discrete minimal common regions (MCRs) of recurrent copy number alterations (CNAs) were identified in genomic DNA from purified plasma cells derived from 65 patients with newly diagnosed multiple myeloma (MM). A total of 14 MCRs, including a deletion at chromosome 17p13.1-17p12 where the TP53 gene resides, were found to be associated with poor survival [2].

In multiple myeloma (MM), TP53 mutations are rare and may represent late events in disease progression [3-7]. The frequency of TP53 deletions detected by fluorescence in situ hybridization (FISH) is reported to range from 9% to 34% in newly diagnosed cases of multiple myeloma and is related to survival [8-13]. However, the role of TP53 loss in the pathogenesis of multiple myeloma, its relationship to gene expression, and its relevance as a prognostic variable, remain to be elucidated.

As a transcription factor, TP53 regulates the expression of genes involved in a variety of cellular functions, including cell-cycle arrest, DNA repair and apoptosis [14-19] but the function of TP53 and the signaling pathways regulated by it in MM are still not clear. The TP53-dependent expression of 122 target genes identified by PET analysis was recently demonstrated [20]. However, expression of only a few of these 122 previously identified TP53 target genes was correlated with TP53 expression in tumor cells from the cohort of 351 MM patients. This suggested that TP53 may regulate a distinct set of genes in MM.

Thus, the prior art is deficient in the knowledge of the relative contribution of TP53 gene status in multiple myeloma. In addition, the prior art is deficient in correlating TP53 gene status with multiple myeloma disease progression and outcome. The present invention fulfills this long-standing need and desire in the art.

### SUMMARY OF THE INVENTION

The present invention is directed to a method for identifying a gene as an independent prognostic factor specific for a disease. Such a method comprises isolating plasma cells from individuals within a population; and extracting nucleic acid from the plasma cells. The extracted nucleic acid is then hybridized to a DNA array to determine expression levels of genes in the plasma cells. Subsequently, multivariate regression analyses on data obtained from the hybridization is performed, where said analysis identifies the gene as an independent prognostic factor specific for the disease.

The present invention is also directed to a method for identifying a gene relevant in prognosis of a disease. Such a method comprises isolating plasma cells from individuals within a population and extracting nucleic acid from the plasma cells. The extracted nucleic acid is then hybridized to a DNA microarray; and a log rank test is performed on the data obtained from the hybridization to identify genes that are up-regulated and down-regulated in the plasma, thereby identifying the gene important for prognosis of the disease.

The present invention is further directed to a method for determining prognosis of an individual with multiple myeloma, comprising: obtaining plasma cells from the individual and determining expression of TP53 alone or in combination with one or more genes selected from the group consisting of TRIM13, NADSYN1, TRIM22, AGRN, CENTD2, SESN1, TM7SF2, NICKAP1, COPG, STAT3, ALOX5, APP, ABCB9, GAA, CEP55, BRCA1, ANLN, PYGL, CCNE2, ASPM, SUV39H2, CDC25A, IFIT5, ANKRA2, PHLDB1, TUBA1A, CDCA7, CDCA2, HFE, RIF1, NEIL3, SLC4A7, FXYD5, MCC, MKNK2, KLHL24, DLC1, OPN3, B3GALNT1, SPRED1, ARHGAP25, RTN2, WNT16, DEPDC1, STT3B, ECHDC2, ENPP4, SAT2, SLAMF7, MAN1C1, INTS7, ZNF600, L3MBTL4, LAPTM4B, OSBPL10, KCNS3, THEX1. CYB5D2, UNC93B1, SIDT1, TMEM57, HIGD24, FKSG44, C14orf28, LOC387763, TncRNA, C18orf1, DCUN1D4, FANCI, ZMAT3, NOTCH1, BTG2, RAB1A, TNFRSF10B, HDLBP, RIT1, KIF2C, S100A4, MEIS1, SGOL2, CD302, C5orf34, FAM111B and C18orf54. The expression level of the gene(s) is then compared with expression level of the gene in a control individual such that genes that are up-regulated, down-regulated or a combination thereof compared to gene expression levels in plasma cell of a control individual indicates prognosis of the individual.

The present invention is still further directed to a kit for prognosis of multiple myeloma, comprising: nucleic acid probes complementary to mRNA of genes described supra; and written instructions for extracting nucleic acid from plasma cells of an individual and hybridizing the nucleic acid to the DNA microarray.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIGS. 1A-1C show that low expression levels of TP53 highly correlated with deletion and adversely affects outcome. In FIG. 1A, the 194 newly diagnosed multiple myeloma on Total Therapy 2 were divided into two groups based on TP53 deletion. Kaplan-Meier estimates showed 5-year actuarial probabilities of (i) 36% being event-free and 52% alive in cases with TP53 deletion and (ii) 51% event-free and 70% alive in those without TP53 deletion (P<0.005). FIG. 1B shows that TP53 deletion is highly correlated with low TP53 expression. TP53 expression levels as measured by Affymetrix microarray signal, relative to TP53 copy number by FISH is displayed. Expression levels in 154 cases with no evidence of deletion (minimum 680; maximum 5,241; median 1,599; mean 1,487) are significantly higher than in 36 cases with monoallelic deletion (minimum 226; maximum 2,600; median 889; mean 1,044) which are higher than in 4 cases with biallelic deletion (minimum 138; maximum 599; median 470; mean 419). FIG. 1C shows that low TP53 gene expression is related to outcome. Samples from 351 patients with newly diagnosed MM on Total Therapy 2 were divided into two groups based on TP53 Affymetrix signal being greater than or less than 733 (lowest 10%). Kaplan-Meier estimates showed 5-year actuarial probabilities of of 28% being event-free and 41% alive in cases with TP53<733 and (ii) 50% event-free and 68% alive in those with TP53>733 (P<0.0005).

FIGS. 2A-2C show influence of TP53 expression on survival in molecular risk disease, post-relapse survival and survival in an independent cohort. FIG. 2A shows that low TP53 expression influences survival in molecular risk classes myeloma. When TP53 expression was put in the context of a recently described risk stratification, Kaplan-Meier estimates of 5-year actuarial probabilities of (i) 34% being event-free and 52% being alive among cases with low risk cases and TP53 signal>733 compared to 55% event-free and 69% alive among cases with low risk and TP53 signal>733 (P<0.005). TP53 expression did not influence survival in high risk disease FIG. 2B shows that low TP53 expression negatively influences post-relapse survival. The 90 patients in Total Therapy 2 with relapsed MM were divided into two groups based in the TP53 Affymetrix signal being less than or greater than 733. Kaplan-Meier estimates show 5-year actuarial probabilities of 14% alive in cases with TP53<733 versus 35% alive in cases with TP53>733 (P<0.05). FIG. 2C shows that low TP53 expression negatively influences survival in an independent cohort. The 214 patients newly diagnosed with MM enrolled on Total Therapy 3 were divided into two groups based on TP53 expression level being less than or greater than 733. Kaplan-Meier estimates show 2-year actuarial probabilities of (i) 63% being event free and (ii) 66% alive among cases with TP53<733 versus 81% event-free and 88% alive among those with TP53 >733 (P<0.05).

FIGS. 3A-3B show that TP53 expression and its associated gene expression in 51 paired MM patients on Total Therapy 2 at baseline and disease relapse. In FIG. 3A, 51 patients had similar TP53 expression levels at baseline and relapse (P=0.455); only eight patients had at least a 2-fold TP53 signal change, five decreased and three increased at relapse. In FIG. 3B, two-dimensional unsupervised hierarchical cluster analysis of 85 TP53-regulated genes in 51 MM patients with paired gene expression data at baseline and relapse shows that 36 of 51 patients have very similar gene expression patterns of the 85 TP53-regulated genes and these genes cluster closely together (marked by red bracket: BL indicates baseline; RL, relapse).

FIGS. 4A-4B show correlation of expression genes with TP53 expression. FIG. 4A shows heatmap of 122 TP53 target genes in 351 newly diagnosed MM patients on Total Therapy 2, 214 patients on Total Therapy 3 and 90 relapsed MM patients on Total Therapy 2. FIG. 4B shows the normalized log ratio of the 122 TP53 target genes in the overexpression experiments involving four MM cell lines.

FIGS. 5A-5C show effect of TP53 expression on MM cell survival. In FIG. 5A, TP53 (in both nuclear and cytosolic extracts) and cleaved PARP (in nuclear extracts) were evaluated by Western blot performed in OCI-MY5 cells after lentiviral infection of TP53 and empty vector (EV) at 4, 8, 12, and 24 hours. Histone H4 and β-tubulin were used as loading controls. FIG. 5B shows effect of overexpression of TP53 on cell viability in JJN3, OCI-MY5, ARP-1, and Delta 47 cells. Cell viability was evaluated by trypan blue exclusion every 12 hours after lentiviral infection of TP53 compared to the EV. FIG. 5C shows that overexpression of TP53 induces apoptosis. Cell cycle distribution and apoptosis were evaluated by flow cytometry performed 24 hours after lentiviral infection in JJN3, OCI-MY5, ARP-1, and Delta 47 cells infected with EV or TP53 cDNA. Note that overexpression of TP53 induced a dramatic increase in the percentage of cells with sub-G₀-phase DNA content (indicative of apoptosis).

FIGS. 6A-6B show gene expression profile of 85 TP53-regulated genes in MM cell lines and patients. A total of 85 genes were up-regulated (n=50) or down-regulated (n=35) at least 1.5-fold in at least three of four MM cell lines, and also exhibited differential expression in a comparison of primary MM between the lowest relative to the highest TP53 expressers. In FIG. 6A, the red in the normalized log ratio (TP53 overexpression vs. empty vector) of the four MM cell lines (JJN3, OCI-MY5, ARP-1, and Delta 47) represents induction, and green represents repression. FIG. 6B shows differences in gene expression between 36 patients on TT2 with lowest TP53 expression and 36 patients with the highest TP53 expression.

FIG. 7 shows networks of TP53-regulated genes in MM cell lines and patients. Ingenuity Pathways Analysis software was used to analyze the identified genes (n=85). Three networks were identified. The network representing proteins involved in the biological functions of cancer and the cell cycle is shown. The genes written in bold letters with a shaded node were identified by microarray analysis, and the other genes were those related to the regulated genes based on the network analysis. The intensity of a node color indicates the degree of up-regulation (red). The meanings of node shapes are indicated in the figure.

FIGS. 8A-8B show networks of TP53-regulated Genes in MM cell lines and patients. Ingenuity Pathways Analysis software was used to analyze the identified genes (n=85). Three networks were identified. The main network was shown in FIG. 7. The genes written in bold letters with a shaded node were identified by microarray analysis, and the other genes were those related to the regulated genes on the basis of the network analysis. The intensity of a node color indicates the degree of up-regulation (red). The meanings of node shapes are indicated in the figure. The network in FIG. 8A represents proteins involved in the biological functions of the cell cycle and cellular movement, assembly, and organization. The network in FIG. 8B represents proteins involved in the biological functions of cell morphology and DNA replication, recombination, and repair.

FIGS. 9A-9C show gene expression profiles of TP53 regulated genes and their clinical relevance. FIG. 9A shows two-dimensional unsupervised hierarchical cluster analysis of 85 (rows) TP53-regulated genes in CD138-enriched plasma cells of newly diagnosed MM patients on TT2 (n=351). The right branch consists of MM samples that have a gene expression profile associated with a high TP53 expression level (horizontal green bar), and the left branch contains MM samples that have a gene expression profile associated with low TP53 expression level (horizontal red bar). Kaplan-Meier estimates of (FIG. 9B) EFS and (FIG. 9C) OS in newly diagnosed MM patients on TT2 show superior 5-year actuarial probabilities of EFS (51% vs. 39%; P=0.0006) and OS (69% vs. 46%; P=0.001) in the right-branch patients whose 85-gene expression profile was associated with a high TP53 expression level.

FIGS. 10A-10B show gene expression profiles of TP53-regulated genes and their clinical relevance. Two-dimensional unsupervised hierarchical cluster analysis of 85 (rows) TP53-regulated genes in CD138-enriched plasma cells from (FIG. 10A, top) patients with relapsed MM on TT2 (n=90) and (Figure B, top) newly diagnosed MM patients on TT3 (n=214). The right branch consists of MM samples that have a gene expression profile associated with high TP53 expression (horizontal green bar), and the left branch contains MM samples that have a gene expression profile associated with low TP53 expression (horizontal red bar). (FIG. 10A shows Kaplan-Meier estimates of post-relapse survival in (FIG. 10A, bottom) 90 relapsed-MM patients on TT2 showed superior 5-year actuarial probabilities of post-relapse survival (55% vs. 17%; P<0.0001) and in (FIG. 10A, top) the right-branch patients with an 85-gene expression profile associated with high TP53 expression. FIG. 10B shows Kaplan-Meier estimates of (bottom, right side) EFS and (bottom, left hand side) OS in newly diagnosed MM patients on TT3 showed superior 3-year actuarial probabilities of EFS (88% vs. 68%; P=0.0018) and OS (89% vs. 78%; P=0.0625) in (bottom, right hand side) the right-branch patients with an 85-gene expression profile associated with high TP53 expression.

### DETAILED DESCRIPTION OF THE INVENTION

Powerful prognostic models in MM based on the expression of 17 genes have been described [1]. This risk-stratification model for newly diagnosed MM treated with high-dose chemotherapy was also predictive of the outcome of treating relapsed disease with the single agent bortezomib. The high-risk index based on this model is an extremely powerful prognostic factor with a hazard ratio in excess of 3 [1]. TP53 gene expression, however, was not included in the model.

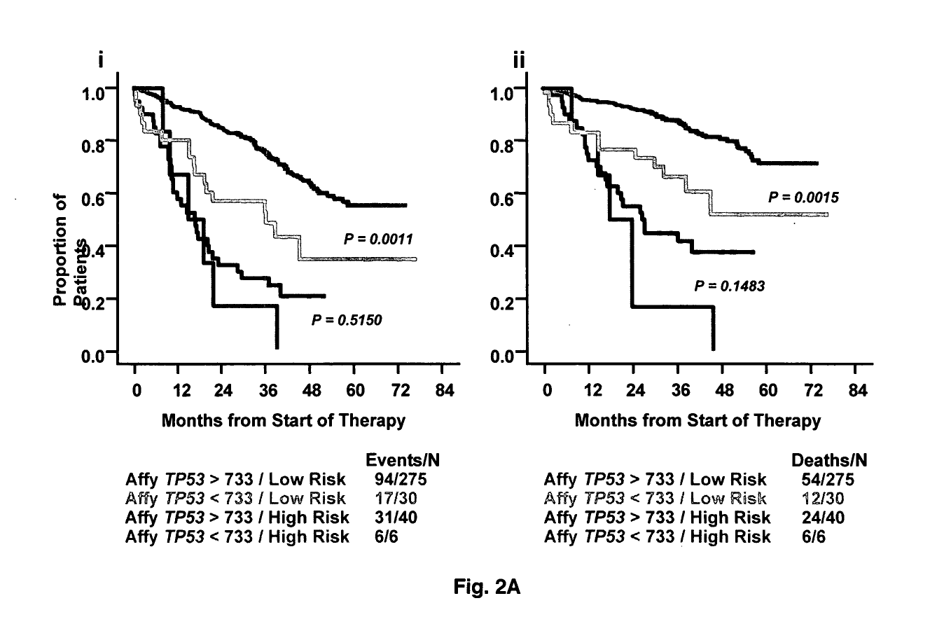

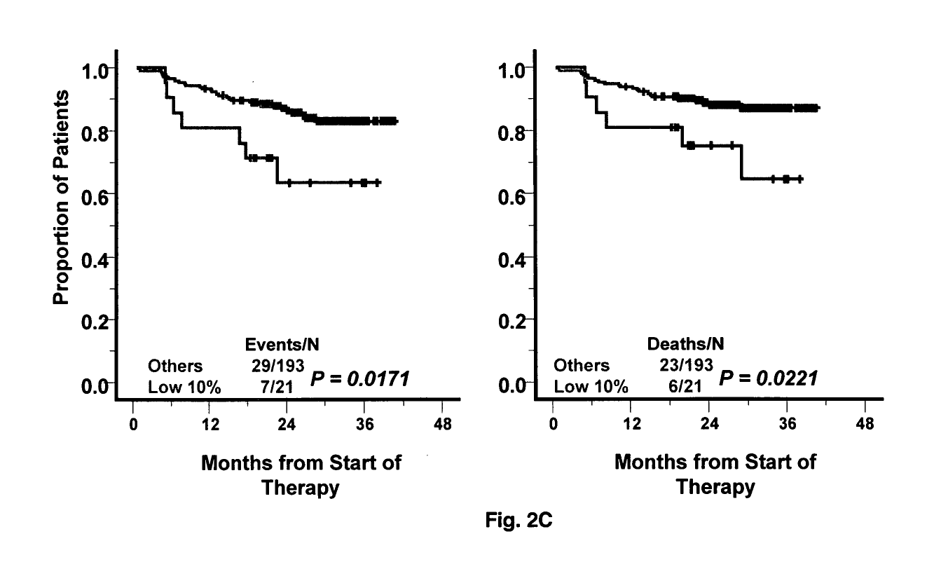

It was observed herein that with a 10% cut-off point (rather than the 25% and 75% cut-off points used to identify the genes in our recent expression-based model of high risk), patients with tumors with TP53 expression levels in the lowest 10^{th} percentile had a significantly shorter EFS and OS than those in the 90^{th} percentile. The present invention demonstrates that low expression levels of TP53 were correlated with mono- or biallelic deletion of the TP53 locus. Multivariate regression analyses revealed that low TP53 expression was an independent adverse prognostic factor and a parameter for predicting shortened survival in both TT2 and TT3, even in the context of high-risk molecular features. But t(4:14) translocation only significant in TT2 and not retained independent significance in TT3 (Table 1), it may imply that bortezomib can overcome negative impact of t(4:14) but can not overcome low TP53 expression and high risk model [24]. Low TP53 expression was able to further dissect the survival of low-risk patients defined by the 17-gene model (FIG. 2A). One 18 gene test (17 high risk gene and TP53) can provide more prognostic information than all other tests in combination, including standard laboratory, imaging as well as cellular and other molecular genetic parameters. The R² value, a measure of accounting for clinical outcome variability [23], increased from 38.4% to 39.7% (data not shown). These data add to the continued refinement of molecular prognostics in MM.

In addition to identifying TP53 as a poor prognostic factor, this study also provides, for the first time, a comprehensive list of genes that are differentially expressed in association with TP53 expression in MM. The TP53 tumor suppressor gene plays a key role in prevention of tumor formation through transcriptional-dependent and -independent mechanisms. Transcriptional-dependent mechanisms are mainly mediated by TP53 regulation of downstream targets, leading to growth arrest and apoptosis [37]. Recently, a global map of TP53 transcription factor binding sites in the human genome was identified in a colorectal cancer cell line by PET analysis, and 122 TP53 target genes were characterized [20] Their TP53-dependent expression was verified in breast cancer patients [20] however, expression of only a few of these genes was correlated with TP53 expression in MM cells. This suggested that TP53 might regulate a distinct set of genes in MM. Through cross-validation in human MM cell lines and samples from two large cohorts of MM patients, a comprehensive panel of 85 putative targets of TP53 were identified that were correlated with clinical outcome. None of the 85 TP53-associated genes were identified in the previous high-risk 70-gene model [1]. This suggests that TP53 and its associated genes may complement our 70-gene model.

It is noteworthy that 69 of the 85 TP53-regulated genes have a defined function in apoptosis and the cell cycle, DNA repair and chromatin modification, cell growth and differentiation, and transcriptional regulation. Identification and characterization of these genes and their pathways may lead to a better understanding of the critical role of TP53 loss in MM. TP53-induced growth arrest is achieved mainly by transactivation of p21 (for G₁-phase arrest), of 14-3-3σ (for G₂-phase arrest), or of placenta transforming growth factor-β. TP53 regulates apoptosis in transcriptional-dependent and -independent manners. Under a transcriptional-dependent mechanism, TP53 induces apoptosis by transactivating the genes in both mitochondrial and death receptor pathways, as well as transrepressing cellular survival genes [37]. The results of analysis of TP53-regulated genes showed that TP53 up-regulates death receptor pathway apoptotic genes (e.g., TNFRSF10B) and down-regulates cell cycle genes (e.g., BRCA1, cyclin E, S100A4, and CDCs) in MM.

Of the 85 TP53-associated genes, only four genes, TNFRSF10B, NOTCH1, ZMAT3, and TRIM22, were previously identified among the 122 TP53 target genes. Both TNFRSF10B and NOTCH1 gene products are cell membrane proteins. TNFRSF10B, also named KILLER/DR5, is a member of the tumor necrosis factor-receptor superfamily and plays a key role in the death receptor pathway. It is located in a minimal region of loss at8p21.3-p12 in MM [2]. TNFRSF10B is a TP53-inducible receptor for the cytotoxic ligand TNFSF10/TRAIL and induces a caspase-dependent apoptotic pathway [38]. The improved recombinant form of the death ligand TRAIL is not cytotoxic for normal human cells and is a good candidate for the treatment of MM [39]. NOTCH1 functions as a receptor for membrane-bound ligands Jagged1, Jagged2, and Delta1 to regulate cell-fate determination, and affects the implementation of differentiation, proliferation, and apoptotic programs [41]. Recent results show that NOTCH1 signaling is involved in bone marrow stroma-mediated de novo drug resistance in MM [42]. ZMAT3, also named WIG1, is a TP53-regulated gene that encodes a growth inhibitory zinc finger protein [43]. Wig-1 can bind short-interfering/micro RNAs in vitro, which raises the possibility that it is involved in miRNA-mediated regulation of cell growth and survival, acting to promote TP53-induced cell growth arrest and/or apoptosis [44]. TRIM22, and another TRIM/RBCC family member, TRIM13, were identified as associated with TP53 expression in the present invention. The interferon-inducible protein TRIM22 has been identified as a TP53 target gene, with possible involvement in hematopoietic proliferation and differentiation [45]. TRIM13 is one of most likely candidates for tumor suppressor gene for B-cell chronic lymphocytic leukemia [46]. TRIM13 has also been found to exhibit copy number-sensitive expression in MM [2]. The roles of TP53 and these universal target genes in both tumor origin and the tumor response to chemotherapy indicate that these types of studies will be useful in developing a more rational approach to cancer treatments.

No significant differences were found in TP53 deletion and expression at baseline and in relapsed disease in 51 paired samples, and it is noteworthy that most (36 of 51) paired samples had a gene expression pattern similar to that observed when TP53 is expressed. This result may imply that the current treatment for MM has no efficacy in regulating TP53 and expression of its associated genes. The present invention also provides evidence that 90% of TP53 deletions in MM are monoallelic deletions. Furthermore, TP53 mutation is not a frequent event in MM [3-7]. Consistent with previous studies, TP53 mutation was not detected in 24 newly diagnosed patients. Overexpression of TP53 can induce strong apoptosis in vitro. Taken together, the results presented herein may indicate an ideal strategy for induction of apoptosis in apoptosis-resistant cancer cells through the modulation of TP53 or MM-specific TP53 signaling pathways.

In conclusion, the present invention demonstrated that low TP53 gene expression is strongly correlated with 17p13 deletion and is an independent adverse prognostic marker in newly diagnosed MM treated with autotransplantations. In addition, using expression profiling, the present invention identified MM-specific genes associated with TP53 expression in both cultured myeloma cells and primary tumors that correlated with clinical outcome. The data presented herein suggest that low levels of expression of TP53 and its regulated genes are associated with a malignant phenotype in MM, and this finding may provide insight into the molecular mechanisms of MM and may inform possible novel targets for future therapies for MM and other cancers.

In one embodiment of the present invention there is provided a method for identifying a gene as an independent prognostic factor specific for a disease, comprising: isolating plasma cells from individuals within a population; extracting nucleic acid from the plasma cells; hybridizing the nucleic acid to a DNA array to determine expression levels of genes in the plasma cells; and performing multivariate regression analyses on data obtained from the hybridization, where the analysis identifies the gene as an independent prognostic factor specific for a disease. Further, the low expression of the gene may correlate with poor prognosis, deletion in chromosome, decreased gene copy number, or a combination thereof. The prognosis may comprise a shorter event-free and overall survival. Additionally, the deletion may be on chromosome 17p13. Furthermore, the gene identified as an independent prognostic factor specific for a disease may include but is not limited to TP53. In case the gene is TP53, the disease may be cancer, where the cancer may include but is not limited to multiple myeloma.

In another embodiment of the present invention there is provided a method for identifying a gene relevant in prognosis of a disease, comprising: isolating plasma cells from individuals within a population; extracting nucleic acid from the plasma cells; hybridizing the nucleic acid to a DNA microarray; and performing log rank test on the data obtained from the hybridization to identify genes that are up-regulated and down-regulated in the plasma, thereby identifying the gene important for prognosis of the disease. This method may further comprise analyzing nucleic acid obtained from the plasma cells; and performing log rank test on data obtained after analyzing the nucleic acid, where the test correlates the status of the gene with progression and outcome of the disease. The analysis of the nucleic acid may comprise determining mRNA expression of the gene, sequence integrity of the gene, copy number of the gene or a combination thereof.

The method may also further comprise performing gene expression profiling to identify genes associated with the gene linked to survival specific for the disease. Examples of the genes thus, identified may include but are not limited to the ones selected from the group consisting of TRIM13, NADSYN1, TRIM22, AGRN, CENTD2, SESN1, TM7SF2, NICKAP1, COPG, STAT3, ALOX5, APP, ABCB9, GAA, CEP55, BRCA1, ANLN, PYGL, CCNE2, ASPM, SUV39H2, CDC25A, IFIT5, ANKRA2, PHLDB1, TUBA1A, CDCA7, CDCA2, HFE, RIF1, NEIL3, SLC4A7, FXYD5, MCC, MKNK2, KLHL24, DLC1, OPN3, B3GALNT1, SPRED1, ARHGAP25, RTN2, WNT16, DEPDC1, STT3B, ECHDC2, ENPP4, SAT2, SLAMF7, MAN1C1, INTS7, ZNF600, L3MBTL4, LAPTM4B, OSBPL10, KCNS3, THEX1. CYB5D2, UNC93B1, SIDT1, TMEM57, HIGD24, FKSG44, C14orf28, LOC387763, TncRNA, C18orf1, DCUN1D4, FANCI, ZMAT3, NOTCH1, BTG2, RAB1A, TNFRSF10B, HDLBP, RIT1, KIF2C, S100A4, MEIS1, SGOL2, CD302, C5orf34, FAM111B and C18orf54. Moreover, the method may correlate the expression of the gene to survival of an individual suffering from the disease, with molecular classification of the disease, with molecular risk stratification of the disease to predict outcome or a combination thereof. Additionally, the low expression of the gene may correlate with the high-risk molecular classification of the disease. Further, the high-risk molecular classification of multiple myeloma maybe characterized by increased combined expression of MMSET, MAF/MAFB and PROLIFERATION signatures. Furthermore, the prognosis may comprise a shorter event-free and overall survival. Example of the gene identified by such a method may include but is not limited to TP53 and the disease may be cancer. Example of the cancer may include but is not limited to multiple myeloma.

In yet another embodiment of the present invention, there is a method for determining prognosis of an individual with multiple myeloma, comprising: obtaining plasma cells from the individual; determining expression of TP53 alone or in combination with one or more genes selected from the group consisting of TRIM13, NADSYN1, TRIM22, AGRN, CENTD2, SESN1, TM7SF2, NICKAP1, COPG, STAT3, ALOX5, APP, ABCB9, GAA, CEP55, BRCA1, ANLN, PYGL, CCNE2, ASPM, SUV39H2, CDC25A, IFIT5, ANKRA2, PHLDB1, TUBA1A, CDCA7, CDCA2, HFE, RIF1, NEIL3, SLC4A7, FXYD5, MCC, MKNK2, KLHL24, DLC1, OPN3, B3GALNT1, SPRED1, ARHGAP25, RTN2, WNT16, DEPDC1, STT3B, ECHDC2, ENPP4, SAT2, SLAMF7, MAN1C1, INTS7, ZNF600, L3MBTL4, LAPTM4B, OSBPL10, KCNS3, THEX1. CYB5D2, UNC93B1, SIDT1, TMEM57, HIGD24, FKSG44, C14orf28, LOC387763, TncRNA, C18orf1, DCUN1D4, FANCI, ZMAT3, NOTCH1, BTG2, RAB1A, TNFRSF10B, HDLBP, RIT1, KIF2C, S100A4, MEIS1, SGOL2, CD302, C5orf34, FAM111B and C18orf54; and comparing the expression level of the gene(s) with expression level of the gene in a control individual such that genes that are up-regulated, down-regulated or a combination thereof compared to gene expression levels in plasma cell of a control individual indicates prognosis of the individual.

The individual with poor prognosis may have up-regulated expression of one or more genes selected from the group consisting of CEP55, BRCA1, ANLN, PYGL, CCNE2, ASPM, SUV39H2, CDC25A, TUBA1A, CDCA7, CDCA2, HFE, RIF1, NEIL3, SLC4A7, OPN3, B3GALNT1, SPRED1, DEPDC1, ENPP4, INTS7, L3MBTL4, THEX1, DCUN1D4, FANCI, ZMAT3, NOTCH1, BTG2, RAB1A, TNFRSF10B, HDLBP, RIT1, KIF2C, S100A4, MEIS1, SGOL2, CD302, C5orf34, FAM111B and C18orf54 and may have down-regulated expression of TP53 alone or in combination with one or more genes selected from the group consisting of TRIM13, NADSYN1, TRIM22, AGRN, CENTD2, SESN1, TM7SF2, NICKAP1, COPG, STAT3, ALOX5, APP, ABCB9, GAA, IFIT5, ANKRA2, PHLDB1, FXYD5, MCC, MKNK2, KLHL24, DLC1, ARHGAP25, RTN2, WNT16, STT3B, ECHDC2, SAT2, SLAMF7, MAN1C1, ZNF600, LAPTM4B, OSBPL10, KCNS3, CYB5D2, UNC93B1, SIDT1, TMEM57, HIGD24, FKSG44, C14orf28, LOC387763, TncRNA and C18orf1. The poor prognosis comprises a shorter event-free and overall survival, a high-risk subtype of the multiple myeloma or both. The high-risk subtype of multiple myeloma is further characterized by increased combined expression of MMSET, MAF/MAFB and PROLIFERATION signatures. The gene expression in such a case may be determined by RT-PCR or DNA microarray. Further, the control individual may be a normal healthy individual.

In still yet another embodiment, there is a kit for prognosis of multiple myeloma, comprising: nucleic acid probes complementary to mRNA of genes described supra; and written instructions for extracting nucleic acid from plasma cells of an individual and hybridizing said nucleic acid to the DNA microarray.

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

### Study Subjects

Purified plasma cells (PCs) were obtained from newly diagnosed multiple myeloma patients who were treated on NIH-sponsored clinical trials UARK 98-026 (Total Therapy 2, TT2) (n=351) and UARK 03-033 (Total Therapy 3, TT3) (n=214) [1, 21-26]. Both protocols utilized induction regimens, followed by melphalan-based tandem autotransplants, consolidation chemotherapy and maintenance treatment.

Human MM cell lines ARP-1, JJN3, OCI-MY5 and Delta 47 were cultured in RPMI 1640 containing 10% heat-inactivated fetal calf serum (FCS), 2 mM L-glutamine (Gibco, Grand Island, N.Y.), penicillin (100 U/mL) and streptomycin (100 μg/mL) at 37° C. in humidified 95% air and 5% CO₂.

### EXAMPLE 2

Fluorescence in situ Hybridization

To detect TP53 deletions, a SpectrumRed-labeled DNA probe (LSI p53; Vysis, Downers Grove, Ill.) was combined with a SpectrumGreen-labeled probe (CEP17, Vysis) for the chromosome 17 α-satellite-DNA centromere. The triple color interphase (TRI)-FISH procedure used to analyze the samples has been described [27, 28]. Based on FISH studies of normal bone marrow mononuclear cells, the upper limit of normal plus three standard deviations was less than 10% for deletions of TP53;[8] therefore, the background cut-off level of 10% was used for the probes sets.

### EXAMPLE 3

### Gene Expression Profiling

Bone marrow plasma cells from 565 newly diagnosed (351 TT2 and 214 TT3) patients, and 90 patients with relapsed disease were purified by CD138 (+) selection (29, 30). Gene expressions levels in purified plasma cells and MM cell lines were profiled using with the U133plus2.0 array (Affymetrix, Santa Clara, Calif.), and the signals of probe set 201746 at representing TP53 was used in this analysis. Signal intensities were preprocessed using GCOS1.1 software and normalized by GCOS1.1 software [29-31]. Gene expression data on this patient cohort can be found at the NIH GEO omnibus under accession number GSE2658 [1,21,22,25,27].

### EXAMPLE 4

### Over-Expression of the TP53 Gene in MM Cell Lines

The amplified TP53 cDNA sequence was cloned into the pWPI lentiviral vector, which was a generous gift from Didier Trono, MD (National Center for Competence in Research, Lausanne, Switzerland) [32]. Recombinant lentivirus was produced by transient transfection of 293T cells according to a standard protocol [33,34]. Crude virus was concentrated by ultracentrifugation at 26,000 rpm for 90 minutes. Viral titers were determined by measuring the amount of HIV-1 p24 antigen by enzyme-linked immunosorbent assay (ELISA) (NEN Life Science Products, Boston, Mass.). A 99% transduction efficiency of MM cell lines was achieved with 3000 ng of lentiviral p24 particles per 10⁶ cells.

### EXAMPLE 5

### Western Blotting

To test TP53 protein levels in MM cell lines and for TP53 over-expression studies, nuclear protein was isolated with the Nuclear/Cytosol Fractionation Kit (BioVision Research Products, Mountain View, Calif.). Nuclear protein (30 μg) was separated by electrophoresis on 4-12% SDS-polyacrylamide gels, and Western blotting was performed with the WesternBreeze Chemiluminescent Immunodetection protocol (Invitrogen, Carlsbad, Calif.). Antibodies to anti-poly(ADP-ribose) polymerase (PARP), anti-β-tubulin, and anti-histone 4 were purchased from Upstate Biotechnology (Charlottesville, Va.); anti-p53 was purchased from Chemicon International (Temecula, Calif.).

### EXAMPLE 6

### Evaluation of DNA Binding Activity of TP53 by ELISA

The DNA binding activity of TP53 was quantified by ELISA using the TransAM p53 transcription factor assay kit (Active Motif North America, Carlsbad, Calif.) according to the manufacturer's instructions. Briefly, nuclear extracts were prepared as described [33] and incubated in 96-well plates coated with immobilized oligonucleotide (5′-RRRCWWGYYY-3′, R=A or G, Y=C or T, W=A or T; SEQ ID NO: 1) containing a consensus binding site for TP53. TP53 binding to the target oligonucleotide was detected by incubation with a primary antibody specific for TP53, visualized with anti-IgG-horseradish peroxidase conjugate and developing solution, and quantified at 450 nm with a reference wavelength of 655 nm. Background binding was subtracted from the value obtained for binding to the consensus DNA sequence. Each sample was analyzed in duplicate, and the results were expressed as the mean±SEM.

### EXAMPLE 7

### Cell Cycle/DNA Content Analysis

Cells (1×10⁶) from each sample were fixed in 75% ethanol at −20° C. overnight. The next day, the cells were washed with cold phosphate-buffered saline, treated with 100 μg RNase A (Qiagen, Valencia, Calif.), and stained with 50 μg of propidium iodide (Roche Applied Science, Indianapolis, Ind.). Flow cytometric acquisition was performed with a three-color FACScan flow cytometer and CellQuest software (Becton Dickinson, San Jose, Calif.). For each sample, 10,000 events were gated. Data were analyzed with Modfit LT software (Verity Software House, Topsham, Me.).

### EXAMPLE 8

### TP53 Mutations Detected by Sequencing Analysis

Mononuclear cells were obtained from bone marrow specimens and enriched using a Ficoll-gradient centrifugation method. Genomic DNA was used as a template (100 ng/reaction) for PCR analysis using intronic primer pairs (TP53 Ex2-4-F and TP53 Ex7-9-R) covering exons 2-9 of the TP53 gene, where most TP53 mutations were detected [35]. Sequencing primers nested within the PCR products (TP53-Ex2-4-F: 5′-CAGCCATTCTTTTCCTGCTC-3′ (SEQ ID NO: 2), TP53-Ex2-4-R: 5′-AGGGTGTGATGGGATGGATA-3′ (SEQ ID NO: 3), TP53-Ex5-6-F: 5′-GTTTCTTTGCTGCCGTCTTC-3′ (SEQ ID NO: 4), TP53-Ex5-6-R: 5′-TTGCACATCTCATGGGGTTA-3′ (SEQ ID NO: 5), TP53-Ex7-9-F: 5′-GGAGGCTGAGGAAGGAGAAT-3′ (SEQ ID NO: 6) and TP53-Ex7-9-R: 5′-TTGAAAGCTGGTCTGGTCCT-3′ (SEQ ID NO: 7)).

### EXAMPLE 9

### Statistical Analyses

The Kaplan-Meier method was to estimate OS. OS was defined as the time from the date of registration until death from any cause; survivors were censored at the time of last contact. Significance analysis of microarray (SAM) [36] was used to determine statistically significant expression changes of genes in high- and low-TP53-expressing MM plasma cells. Univariate and multivariate analyses of prognostic factors were performed with the Cox regression.

### EXAMPLE 10

Low TP53 Expression, Highly Correlated with Deletion, is a Significant and Independent Adverse Prognostic Factor in Newly Diagnosed MM

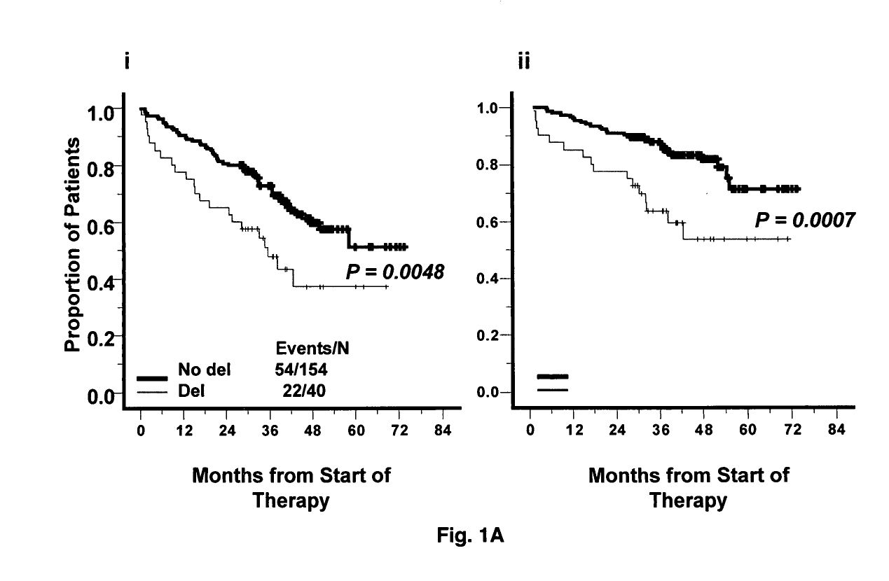

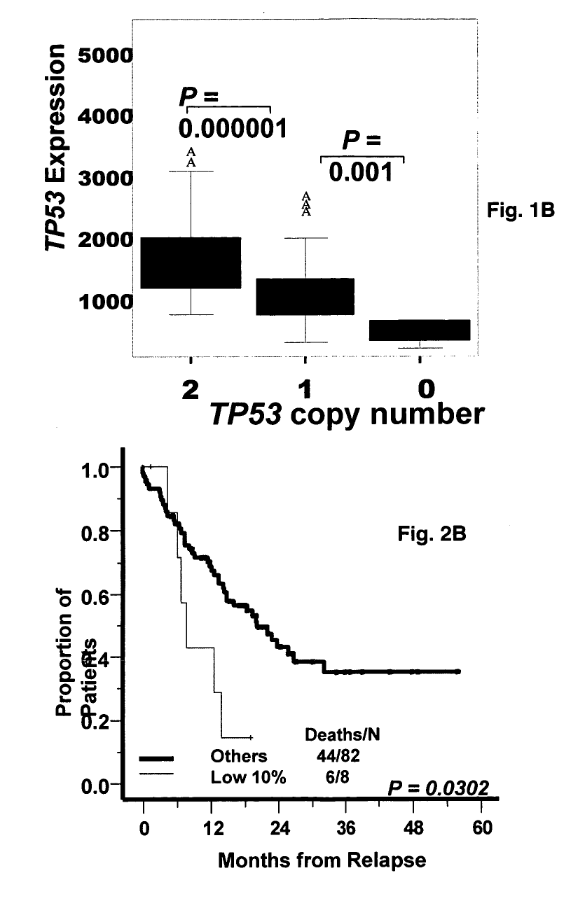

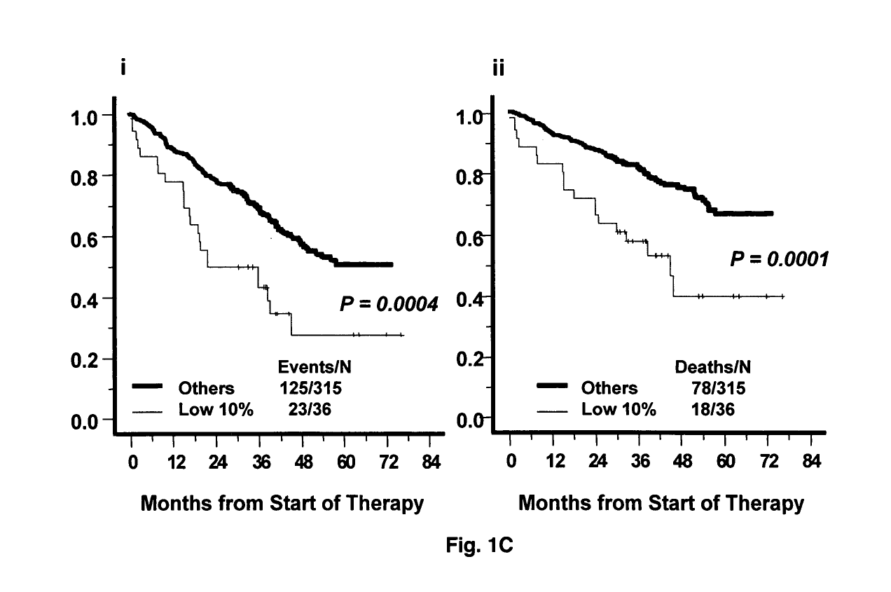

FISH analyses for TP53 deletion were available for 194 TT2 cohort patients with newly diagnosed disease. TP53 deletion was observed in 40 (20.6%) samples, four of which had biallelic deletion. Patients with TP53 deletion were associated with shorter EFS and OS (P=0.0233 and P=0.0007, respectively; FIG. 1A, RHS and LHS). However, increased incidence of deletion was not found in 28 cases for which a sample was tested at diagnosis and at disease relapse paired patients with relapsed disease (data not shown).

TP53 deletion was highly correlated with low TP53 expression. Comparison of TP53 expression level on the basis of deletion status revealed that TP53 expression was lower in 36 monoallelic deletion cases (P<0.001) and even lower in four biallelic deletion cases (P=0.001) than in 150 nondeletion cases (FIG. 1B).

TP53 expression in the 351 newly diagnosed cases varied from an Affymterix signal output (a quantitative measure of the level of activity of a given gene) from a low of 10 to a high of 5,241. Using a running log-rank test, a 10% cutoff was defined as those cases with a TP53 expression level lower than 733 on the basis of the Affymetrix microarray signal represented by 36 of 351 patients with newly diagnosed disease. Genes with an expression level below 500 typically have an absent-detection call and are not detectable by sensitive quantitative RT-PCR. The cases with low TP53 expression were associated with a shorter EFS and OS (P=0.0004 and P=0.0001, respectively; FIG. 1C, right and left hand sides).

With regard to clinical and biological features, patients with a low TP53 expression level had high levels of lactate dehydrogenase (LDH) (P=0.036), increased numbers of bone lesions on magnetic resonance imaging (MRI) (P=0.012), and an increased incidence of deletion of chromosome 13 (P<0.001) and amplification of chromosome 1q21 (P=0.002) (Table 1). In the context of a recently defined molecular subgroup classification [25], the proportion of cases with low levels of TP53 expression was greater in the high-risk molecular subgroups than in the low-risk subgroups. The high-risk molecular subgroups included the MMSET (MS) subtype with a t(4;14) translocation, the MAF/MAFB (MF) subtype with a t(14;16) or a t(14;20) translocation, and the proliferation (PR) subtype; the low-risk groups consisted of subtypes designated hyperdiploid (HY) or low bone disease (LB) or marked by CCND1/CCND3 spike signatures (CD-1 or CD-2) (59% vs. 35%; P=0.023). In the context of molecular risk stratification based on 17 genes [1] TP53 Affymetrix signal<733 was seen in 30 (9.8%) of 305 low-risk and in 6 (13%) of 46 high-risk-disease cases. Low TP53 expression adversely affected both EFS and OS in low-risk but not high-risk disease (FIG. 2A, right and left hand sides). Given the strong correlation between low TP53 expression and high-risk MM subtypes, whether low TP53 expression levels simply reflected the poor prognostic features of high-risk MM [1] or whether it held independent prognostic significance was investigated herein. In a multivariate analysis, low TP53 gene expression was an independent poor-prognostic factor with respect to both EFS and OS (Table 2). Thus, although associated with a number of high-risk-MM features, reduced TP53 gene expression independently confers a poor clinical outcome.

With the same cut-off point, a low TP53 gene expression level predicted short post-relapse survival (P=0.0302; FIG. 2B) in 90 TT2 patients with relapsed disease and short EFS and OS (P=0.0171 and P=0.0221, respectively; FIG. 2C, right and left hand sides) in a separate cohort of 214 patients treated on the successor protocol TT3.

TABLE 1
Baseline patient characteristics according to TP53 expression level

| | Low | | |
|---|---|---|---|
| | TP53 | High | |
| | (%) | TP53 (%) | |
| Characteristics | (n = 36) | (n = 316) | P† |
| Age ≧ 65 yr | 29 | 22 | NS |
| Female sex | 51 | 42 | NS |
| Caucasian race | 94 | 88 | NS |
| IgA isotype | 20 | 26 | NS |
| Creatinine at least 2.0 mg/dl (221 umol/liter) | 20 | 10 | NS |
| MRI focal bone lesions, at least 3 | 77 | 55 | NS |
| CRP at least 8.0 mg/liter | 49 | 34 | NS |
| LDH at least 190 IU/liter | 49 | 31 | .036 |
| β₂-microglobulin | 60 | 59 | |
| Less than 3.5 mg/L | | | |
| At least 3.5 and less than 5.5 mg/L | 14 | 20 | NS |
| ≧5.5 mg/L | 26 | 21 | |
| ALB less than 3.5 g/dl | 17 | 14 | NS |
| Hb less than 10 g/dl | 23 | 26 | NS |
| BMPC (by aspiration) 33% or greater | 61 | 66 | NS |
| Chromosomal abnormalities (defined by G- | 40 | 34 | NS |
| banding) | | | |
| Deletion of chromosome 13 | 84 | 46 | <.001 |
| Amplification of 1q21 | 75 | 42 | .002 |
| High risk model (17 gene)* | 19 | 14 | NS |
| Subgroups with poor prognosis* | 59 | 35 | .023 |
| (Proliferation/MMSET/FGFR3/MAF/MAFB) | | | |

Abbreviations:
MRI, magnetic resonance imaging;
CRP, C-reactive protein;
LDH: lactate dehydrogenase;
NS: not significant.
*High risk model [1] and PR/MS/MF subgroup designation [25] have been described else where.
†Chi-square was used to compare the clinical and biological parameters between cases with the lowest 10% of TP53 expression and the other 90% of cases with higher expression levels.

TABLE 2
Multivariate analysis of clinical characteristics affecting OS and EFS.

| | | | Overall Survival | | Event-Free Survival | |
|---|---|---|---|---|---|---|
| Variable | | n/N (%) | HR (95% CI) | P | HR (95% CI) | P |
| TT2 | Creatinine >= 2.0 mg/dL | 37/334 (11%) | 1.75 (1.07, 2.84) | 0.024 | 1.70 (1.19, 2.43) | 0.004 |
| | LDH >= 190 U/L | 114/334 (34%) | 1.81 (1.24, 2.66) | 0.002 | 1.44 (1.13, 1.82) | 0.003 |
| | Cytogenetic | 108/334 (32%) | 1.77 (1.20, 2.62) | 0.004 | NS | NS |
| | abnormalities | | | | | |
| | Randomization to | 166/334 (50%) | NS | NS | 0.75 (0.60, 0.93) | 0.009 |
| | Thalidomide | | | | | |
| | t(4; 14) | 47/334 (14%) | 1.81 (1.15, 2.85) | 0.010 | 1.87 (1.35, 2.57) | <.001 |
| | 17 gene-defined | 50/334 (15%) | 2.47 (1.58, 3.85) | <.001 | 2.15 (1.56, 2.96) | <.001 |
| | GEP high-risk | | | | | |
| | TP53 high-risk | 35/334 (10%) | 2.01 (1.22, 3.30) | 0.006 | 1.44 (1.00, 2.07) | 0.049 |
| TT3 | Age >= 65 | 50/176 (28%) | 2.32 (1.04, 5.22) | 0.041 | NS | NS |
| | B2M > 5.5 mg/L | 38/176 (22%) | NS | NS | 3.33 (1.68, 6.62) | <.001 |
| | Creatinine >= 2.0 mg/dL | 58/176 (33%) | 3.54 (1.54, 8.16) | 0.003 | NS | NS |
| | Cytogenetic | 30/176 (17%) | 2.42 (1.08, 5.39) | 0.031 | NS | NS |
| | abnormalities | | | | | |
| | 17-gene High-risk | 20/176 (11%) | 3.19 (1.32, 7.68) | 0.010 | 3.97 (1.98, 7.97) | <.001 |
| | TP53 high-risk | 50/176 (28%) | 2.32 (1.04, 5.22) | 0.041 | 2.51 (1.13, 5.57) | 0.024 |

The multivariate model uses stepwise selection with entry level 0.1 and variable remains if meets the 0.05 level. A multivariate P value greater than 0.05 indicates a variable forced into the model, with significant variables chosen by stepwise selection.
HR indicates hazard ratio;
95% CI, 95% confidence interval;
P, probability value from Wald Chi-square test in Cox regression;
NS, not statistically significant at the 0.05-level on multivariate analysis;
LDH, lactate dehydrogenase;
GEP, gene expression profile.
PI, proliferation index [25],
*17 gene-defined GEP high-risk has been described elsewhere.¹
†Variables for which P > 0.05: age, race, sex, isotype, hemoglobin, C-reactive protein, MRI lesions, and albumin

### EXAMPLE 11

### No Significant Increase in Deletion of TP53 or Decrease in Expression of TP53 in Relapsed Disease

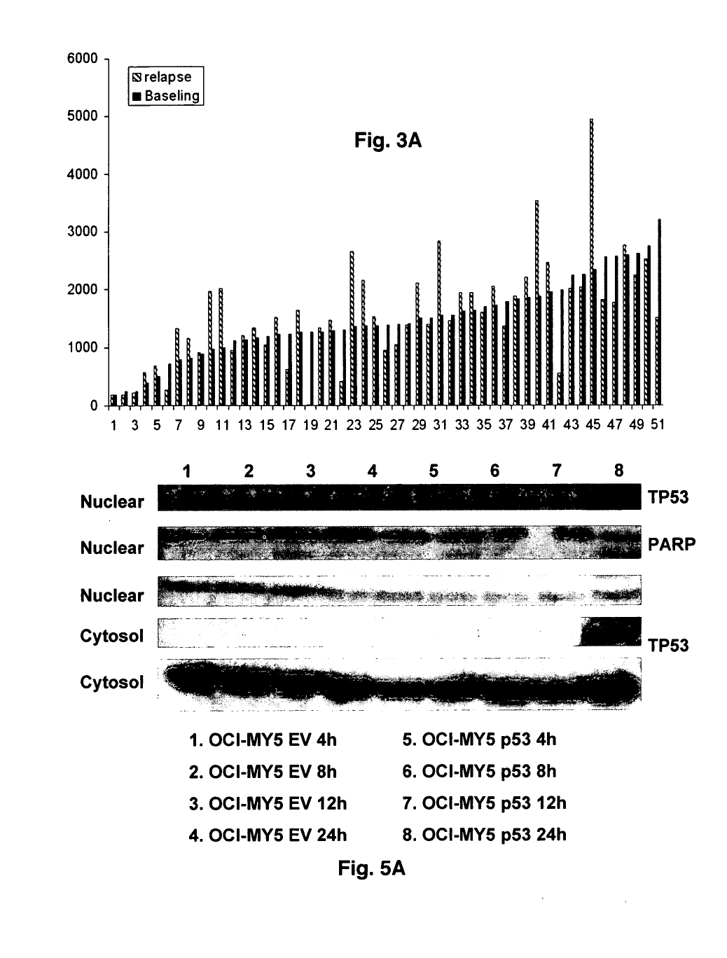

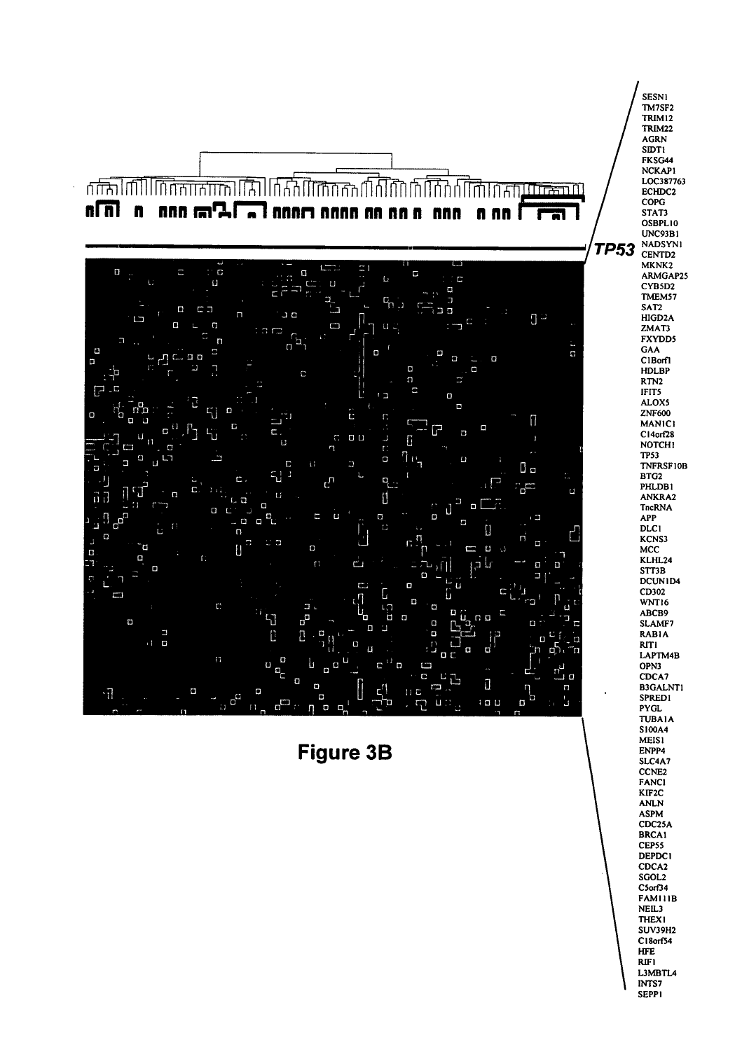

Fifty-one patients had TP53 gene expression data available at both diagnosis and relapse. Consistent with paired FISH results, in these 51 patients, there were also no significant differences in TP53 gene expression at baseline compared with expression at relapse, only eight patients have at least a 2-fold change in TP53 expression level, five having a decreased level and three an increased level at relapse (FIG. 3A).

Interestingly, 36 of 51 patients had very similar gene expression patterns of the 85 TP53-associated genes at baseline and relapse. When the gene expression data on the 51 paired baseline and relapse cases were combined and unsupervised hierarchical clustering was performed, the data clustered closely together (FIG. 3B).

### EXAMPLE 12

### TP53 Mutation is a Rare and Late Event in MM

TP53 mutations were detected by sequencing exons 2-9 in 44 patients, 24 of whom had newly diagnosed disease and 27 had relapsed disease; in 7 of the 44 cases, there were paired baseline and relapse samples. No mutations were detected in 24 newly diagnosed cases or the seven paired baseline-relapse samples, whereas mutations were detected in exons 7, 8 and 9 in 5 of 20 unpaired relapsed-disease samples.

### EXAMPLE 13

### Effects of Overexpressing TP53 on MM Cell Growth and Survival

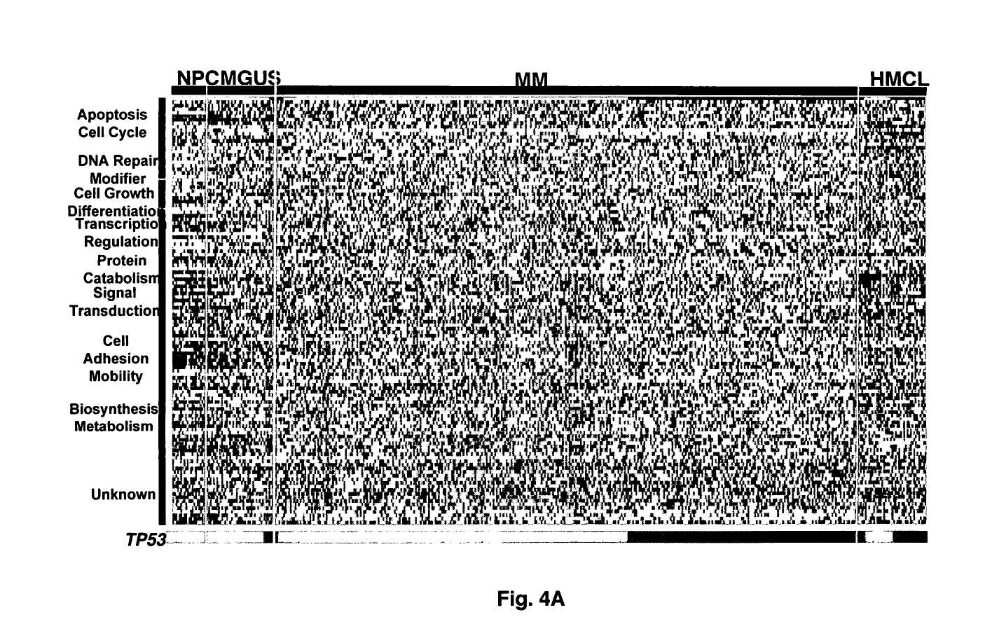

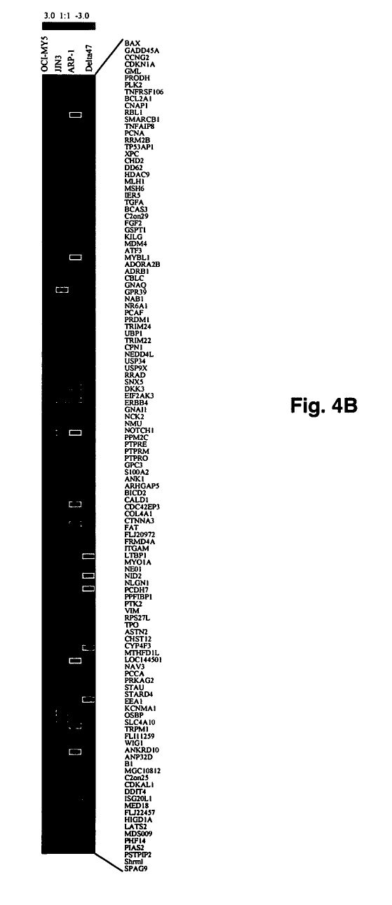

Recently, PET analysis in a colorectal cancer cell line identified 122 TP53 target genes, whose TP53-dependent expression was verified in breast tumors [20]. However, expression of only a few of the previously identified TP53 target genes was correlated with TP53 expression in MM cells (FIG. 4A). This suggested that TP53 might regulate a distinct set of genes in MM. To elucidate the TP53 regulatory networks in MM, lentiviral transduction was used to overexpress TP53 in four MM cell lines: OCI-MY5, JJN3, ARP-1 and Delta 47.

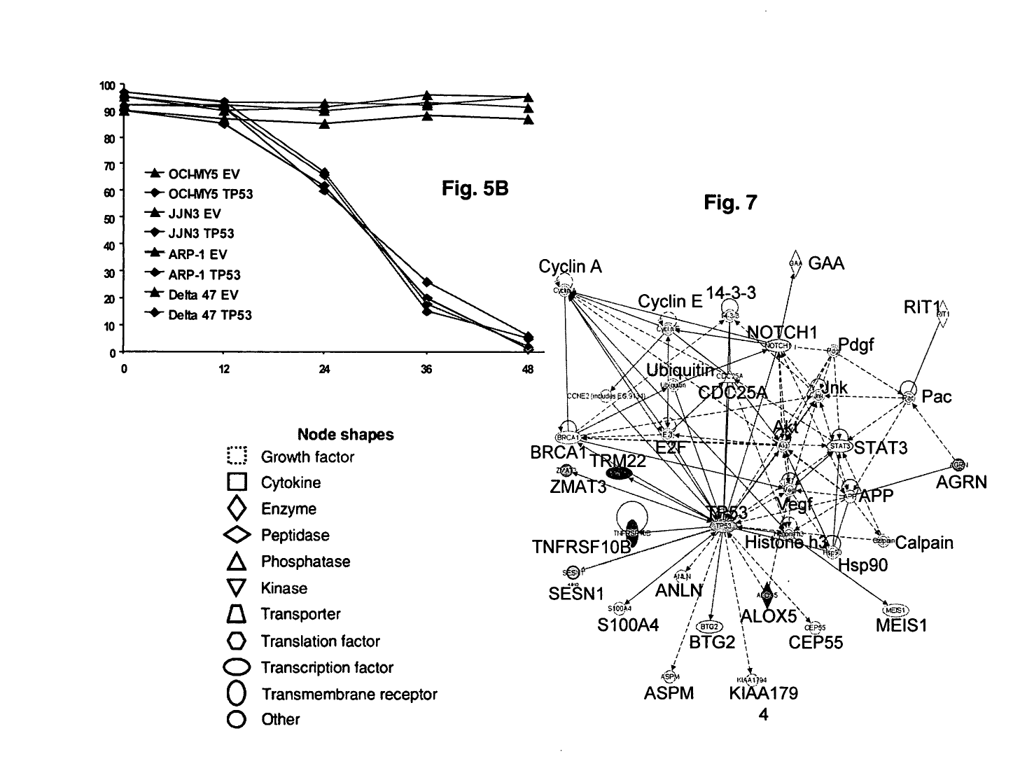

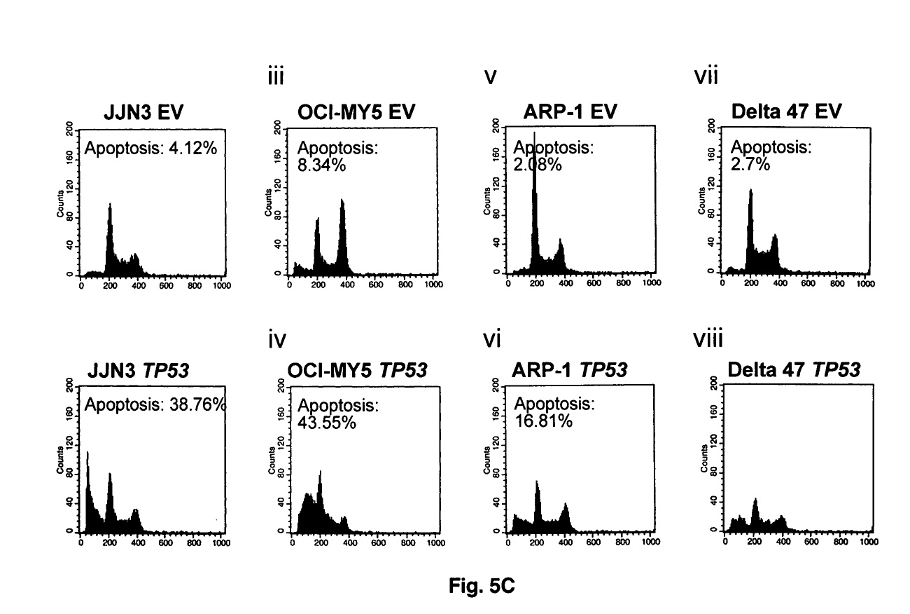

Stable expression of TP53 in OCI-MY5 cells was confirmed by Western blot 24 hours post-lentiviral infection (FIG. 5A). To verify that TP53 was capable of activating target genes, TP53 DNA binding activity was examined. These studies confirmed that TP53 overexpression was correlated with increased DNA binding activity at 24 hours post-lentiviral infection (Data not shown). The effect of TP53 overexpression on MM cell proliferation and viability was also examined. TP53 overexpression decreased cell viability in the four MM cell lines within 24 hours (viability 60%-67%, measured by trypan blue exclusion), and massive cell death occurred within 36 hours (viability 15%-26%), compared with 90% viability of control MM cells infected with empty vector. At 48 hours post-lentiviral transduction, virtually all cells expressing TP53 had died, while the control cells continued to proliferate (FIG. 5B).

Cell proliferation and apoptosis were quantitatively assessed by flow cytometry. The results showed that TP53 expression induced strong apoptosis at 24 hours after infection (FIG. 5C). Analysis of apoptotic mechanisms revealed that TP53 overexpression in MM cells was also associated with cleavage of PARP, an apoptotic marker (FIG. 5A).

On the basis of analysis of protein expression and DNA binding activity, TP53-regulated genes expressed 24 hours post-lentiviral infection were identified herein. Additionally, gene expression profiling showed that at 24 hours there were significantly increased numbers of probe sets, which had a 1.5-fold or greater change between TP53-expressing and -nonexpressing OCI-MY5 cells (data not shown). Therefore, gene expression was profiled in the four MM cell lines (JJN3, OCI-MY5, ARP-1 and Delta 47) at 24 hours post-lentiviral infection to identify TP53-regulated genes.

### EXAMPLE 14

Identification and Classification of Genes Associated with TP53 Expression in MM

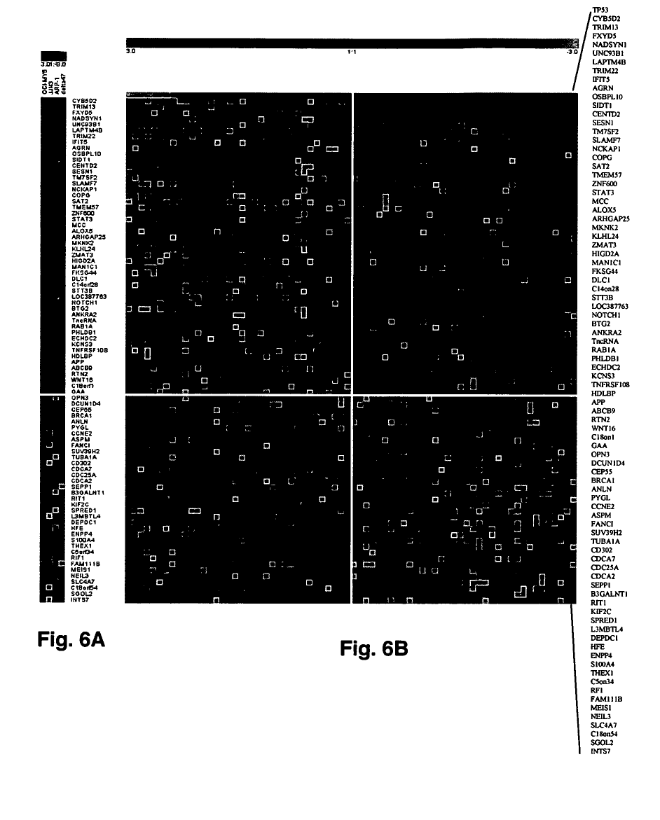

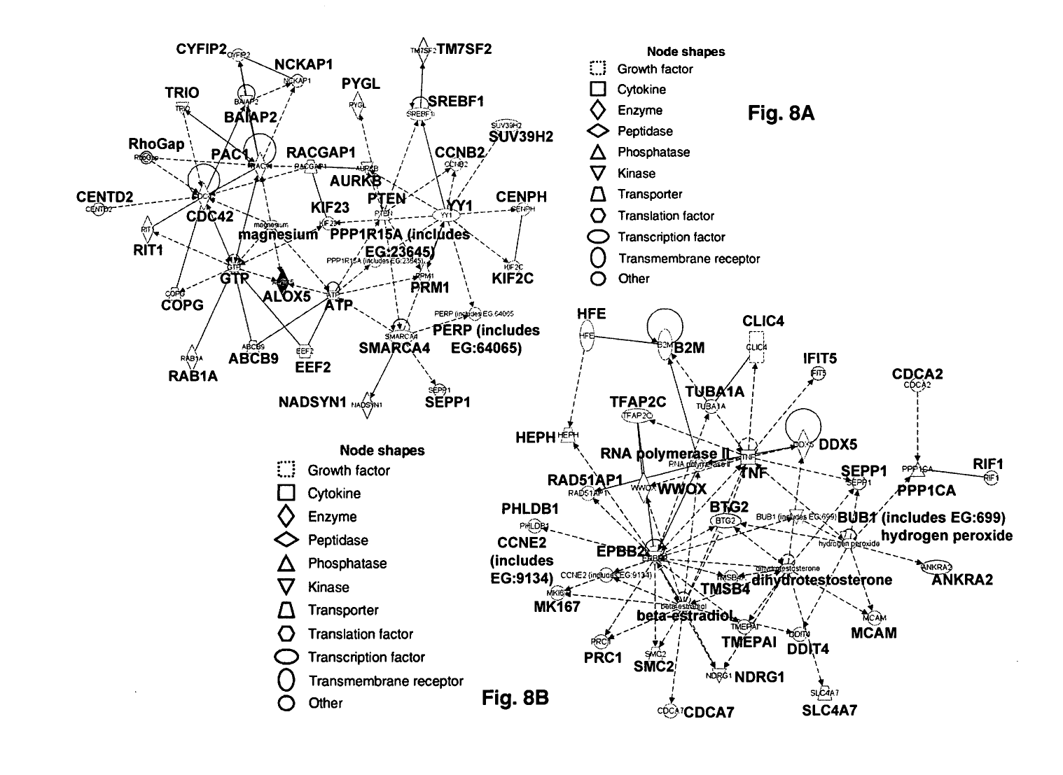

Gene expression profiling revealed that a total of 85 genes were affected by TP53 overexpression (50 being up-regulated and 35 down-regulated) of 1.5-fold or greater in at least three of the four MM cell lines. Consistent with TP53 cellular functions, 69 of the 85 genes in MM were found involved in apoptosis, cell cycle regulation, cell growth and differentiation, DNA repair and chromatin modification, and transcription regulation (Table 3; FIG. 6A). To identify the most relevant biological mechanisms, pathways, and functional categories of the 85 genes affected by TP53 expression, Ingenuity Pathways Analysis software (Ingenuity Systems, Mountain View, Calif.) was used. Three networks were identified, representing proteins involved in cancer and the cell cycle (FIG. 7); cell cycle and cellular movement, assembly, and organization (FIG. 8A); and cell morphology and DNA replication, recombination, and repair (FIG. 8B).

The 85 genes associated with TP53 overexpression also exhibited differential expression in primary MM when the lowest relative to the highest TP53 expressers were compared (FIG. 6B), suggesting that TP53 may directly or indirectly regulate the expression of these genes. None of the differentially expressed genes were identified in our 70-gene high-risk model [1].

From the group of 122 TP53 target genes identified by PET analysis [20], only 11 up-regulated genes were consistently expressed in all four MM cell lines (1.5-fold or greater in at least three of the four MM cell lines; FIG. 4B), and only 4 of these 11 genes (ZMAT3, TNFRSF10B, TRIM22, and NOTCH1) were correlated with TP53 expression in primary MM cells.

TABLE 3
Categories of TP53-associated genes in MM cell lines and newly diagnosis primary tumors

| | | DNA | | | | | | | |
|---|---|---|---|---|---|---|---|---|---|
| | | Repair/ | Cell | | | Post- | | Transport | |
| Apoptosis and | | Chromatin | Growth | Signal | Biosynthesis | translational | Transcription | and Ion | |
| Cell Cycle | | Modifier | Differentiation | Transduction | Metabolism | Modification | Regulation | Channel | Unknown |
| TP53 | APP | IFIT5 | FXYD5 | ARHGAP25 | STT3B | SAT2 | ZNF600 | LAPTM4B | CYB5D2 |
| TRIM13 | ABCB9 | ANKRA2 | MCC | RTN2 | ECHDC2 | SLAMF7 | L3MBTL4 | OSBPL10 | UNC93B1 |
| NADSYN1 | GAA | PHLDB1 | MKNK2 | WNT16 | ENPP4 | MAN1C1 | | KCNS3 | SIDT1 |
| [symbol] | CEP55 | TUBA1A | KLHL24 | DEPDC1 | | INTS7 | | THEX1 | TMEM57 |
| AGRN | BRCA1 | CDCA7 | DLC1 | | | | | | HIGD2A |
| CENTD2 | ANLN | CDCA2 | OPN3 | | | | | | FKSG44 |
| SESN1 | PYGL | HFE | B3GALNT1 | | | | | | C14orf28 |
| TM7SF2 | CCNE2 | RIF1 | SPRED1 | | | | | | LOC387763 |
| NCKAP1 | ASPM | NEIL3 | | | | | | | TncRNA |
| COPG | SUV39H2 | SLC4A7 | | | | | | | C18orf1 |
| STAT3 | CDC25A | | | | | | | | DCUN1D4 |
| ALOX5 | SEPP1 | | | | | | | | FANCI |
| [symbol] | RIT1 | | | | | | | | CD302 |
| [symbol] | KIF2C | | | | | | | | C5orf34 |
| BTG2 | S100A4 | | | | | | | | FAM111B |
| RAB1A | MEIS1 | | | | | | | | C18orf54 |
| [symbol] | SGOL2 | | | | | | | | |
| [symbol] | | | | | | | | | |
| HDLBP | | | | | | | | | |

Up-regulated genes appear in bold, down-regulated genes in plain; previously known TP53 targets are marked by italics and underlining.

### EXAMPLE 15

Clinical Relevance of Genes Associated with TP53 Expression

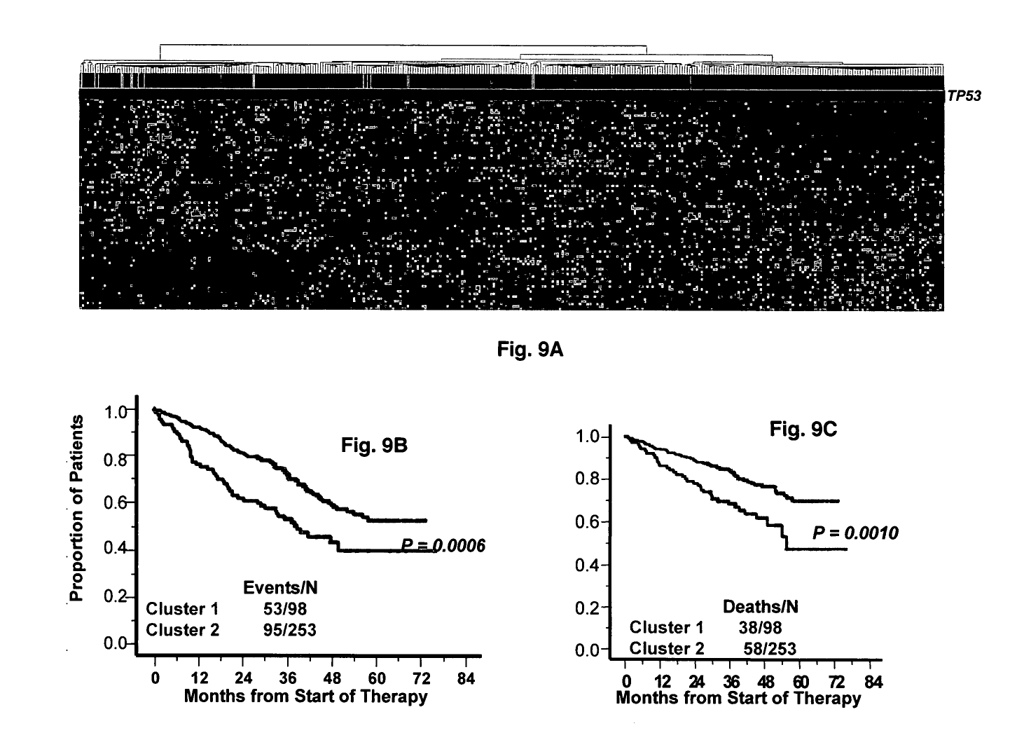

Using the 85 genes identified as associated with TP53 overexpression and the expression data derived from the 351 TT2 patients with newly diagnosed MM, unsupervised hierarchical clustering was performed. This resulted in two primary tumor clusters that were significantly associated with TP53 expression (FIG. 9A). The subtype associated with lower TP53 expression had a significantly shorter EFS (P=0.0006; FIG. 9B) and OS (P=0.0010; FIG. 9C).

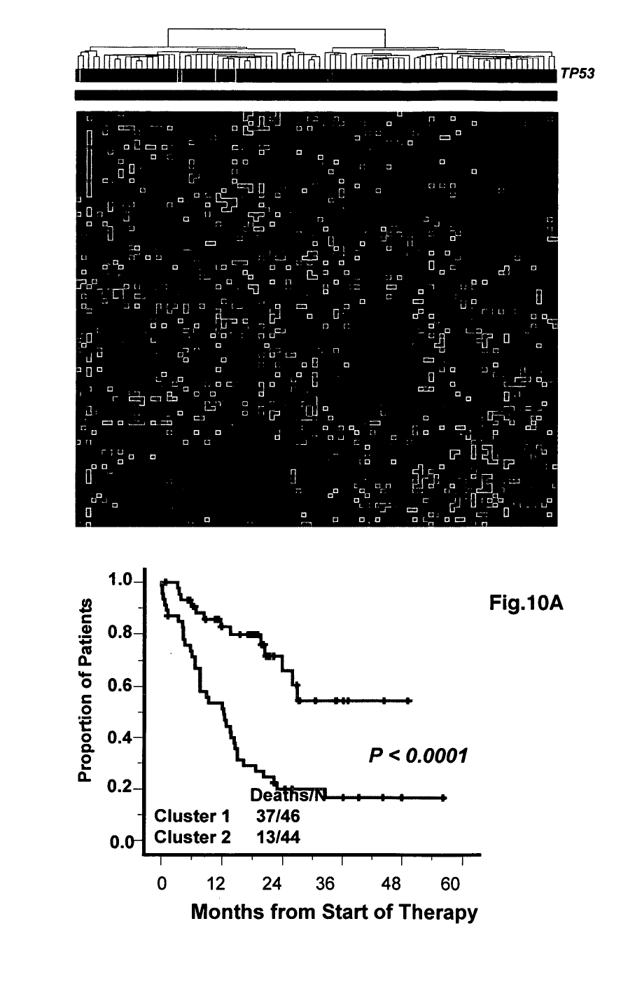

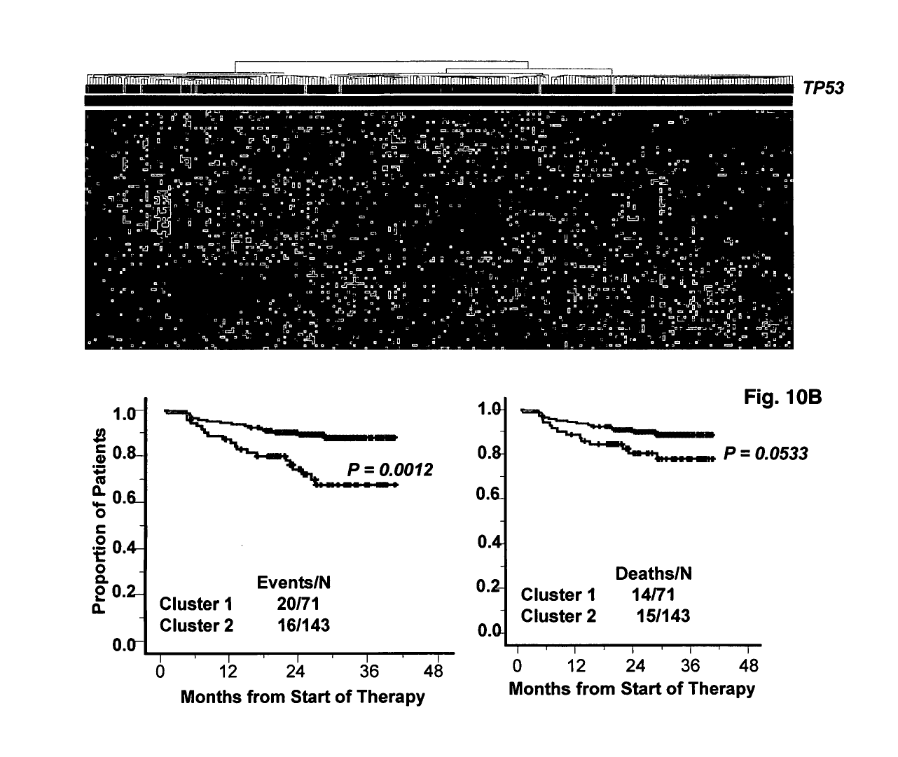

With regard to clinical and biological features, the subtype of patients associated with low TP53 expression had high levels of creatinine (P=0.011) and LDH (P=0.017), low levels of albumin (P=0.041), an increased number of bone lesions on MRI (P=0.001), and an increased incidence of chromosomal abnormalities defined by G-banding (P=0.002), deletion of chromosome 13 (P<0.001), and amplification of chromosome 1q21 (P=0.002) (Table 4). By the same unsupervised hierarchical clustering, the subcluster of MM associated with lower TP53 expression levels had a significantly shorter post-relapse survival (P<0.0001; FIG. 10A) in 90 TT2 cases with relapsed disease and short EFS (P=0.0012) and OS (P=0.0533) in a separate cohort of 214 patients treated on the successor protocol TT3 (FIG. 10B).

Taken together, these findings strongly argue that the 85 novel TP53-regulated genes of MM identified by gene expression profiling in vivo and in vitro are functional in TP53-mediated tumorigenesis and that their expression characteristics in vivo can potentially be used as molecular gauges of tumor aggressiveness and clinical outcome.

TABLE 4
Baseline TT2 patient characteristics according to TP53-regulated gene
cluster.

| | Cluster 1 | | |
|---|---|---|---|
| | (%) | Cluster 2 (%) | |
| Characteristics | (n = 98) | (n = 253) | P† |
| Age at least 65 y | 21 | 22 | NS |
| Female sex | 42 | 44 | NS |
| White race | 91 | 88 | NS |
| IgA isotype | 30 | 24 | NS |
| Creatinine at least 2.0 mg/dL | 19 | 9 | 0.011 |
| MRI focal bone lesions, at least 3 | 72 | 53 | 0.001 |
| C-reactive protein at least 4.0 mg/L | 45 | 32 | 0.023 |
| Lactate dehydrogenase at least | 44 | 30 | 0.017 |
| 190 IU/L | | | |
| β₂-Microglobulin at least 4.0 mg/L | 38 | 33 | NS |
| Albumin less than 3.5 g/dL | 45 | 33 | 0.041 |
| Hemoglobin less than 10 g/dL | 31 | 23 | NS |
| Bone marrow plasma cells (by | 47 | 56 | NS |
| aspiration) at least 33% | | | |
| Chromosomal abnormalities | 48 | 30 | 0.002 |
| (defined by G-banding) | | | |
| Deletion of chromosome 13 | 66 | 43 | <0.001 |
| Amplification of chromosome | 65 | 36 | 0.002 |
| 1q21 | | | |
| High-risk model (17-gene)* | 36 | 7 | <0.001 |
| Subgroups with poor prognosis* | 50 | 51 | NS |
| (Proliferation/MMSET/FGFR3/ | | | |
| MAF/MAFB) | | | |

NS: not significant.
*High-risk model¹ and PR/MS/MF subgroup designation²⁵ are described elsewhere.
†Chi-square was used to compare the clinical and biological parameters between cases in hierarchical cluster 1 (correlated with low TP53 expression) and cluster 2 (correlated with high TP53 expression).

### REFERENCES

Shaughnessy et al. Blood. 2007;109:2276-2284.

Carrasco et al. Cancer Cell. 2006;9:313-325.

Paydas et al. Mol Pathol. 1997;50:329.

Owen et al. Mol Pathol. 1997;50:18-20.

Ollikainen et al. Scand J Clin Lab Invest. 1997;57:281-289.

Yasuga et al. Int J Hematol. 1995;62:91-97.

Neri et al. Blood. 1993;81:128-135.

Chang et al. Blood. 2005;105:358-360.

Ortega et al. Ann Hematol. 2003;82:405-409.

Drach et al. Br J Haematol. 2000;108:886.

Carlebach et al. Cancer Genet Cytogenet. 2000;117:57-60.

Avet-Loiseau et al. Br J Haematol. 1999;106:717-719.

Drach et al. Blood. 1998;92:802-809.

Kho et al. J Biol Chem. 2004;279:21183-21192.

Cawley et al. Cell. 2004;116:499-509.

Kannan et al. Oncogene. 2001;20:3449-3455.

Kannan et al. Oncogene. 2001;20:2225-2234.

Zhao et al. Genes Dev. 2000;14:981-993.

Yu et al. Proc Natl Acad Sci USA. 1999;96:14517-14522.

Wei et al. Cell. 2006;124:207-219.

Zhan et al. Blood. 2007;109:4995-5001.

Zhan et al. Blood. 2007;109:1692-1700.

Shaughnessy et al. Br J Haematol. 2007;137:530-536.

Barlogie et al. Br J Haematol. 2007;138:176-185.

Zhan et al. Blood. 2006;108:2020-2028.

Barlogie et al. N Engl J Med. 2006;354:1021-1030.

Hanamura et al. Blood. 2006;108:1724-1732.

Shaughnessy et al. Blood. 2003;101:3849-3856.

Zhan et al. Blood. 2003;101:1128-1140.

Zhan et al. Blood. 2002;99:1745-1757.

Tian et al. N Engl J Med. 2003;349:2483-2494.

Trono D. J Gene Med. 2000;2:61-63.

Colla et al. Blood. 2007;109:4470-4477.

Zufferey et al. Nat Biotechnol. 1997;15:871-875.

IARC. TP53 mutation database. http://www-p53iarcfr.

Tusher et al. Proc Natl Acad Sci USA. 2001;98:5116-5121.

Sun Y. Mol Carcinog. 2006;45:409-415.

Wu et al. Nat Genet. 1997;17:141-143.

Gazitt Y. Leukemia. 1999; 13:1817-1824.

Gomez-Benito et al. Exp Cell Res. 2007;313:2378-2388.

Qi et al. Cancer Res. 2003;63:8323-8329.

Nefedova et al. Blood. 2004;103:3503-3510.

Hellborg et al. Oncogene. 2001;20:5466-5474.

Mendez Vidal et al. FEBS Lett. 2006;580:4401-4408.

Obad et al. Leuk Res. 2007;31:995-1001.

van Everdink et al. Cancer Genet Cytogenet. 2003;146:48-57.

What is claimed is:

1. A method for identifying a gene as an independent prognostic factor specific for a disease, comprising:
isolating plasma cells from individuals within a population;
extracting nucleic acid from said plasma cells;
hybridizing said nucleic acid to a DNA array to determine expression levels of genes in the plasma cells; and
performing multivariate regression analyses on data obtained from said hybridization, wherein said analysis identifies the gene as an independent prognostic factor specific for a disease.

2. The method of claim 1, wherein the low expression of said gene correlates with poor prognosis, deletion in chromosome, decreased gene copy number or a combination thereof.

3. The method of claim 2, wherein said prognosis comprises a shorter event-free and overall survival.

4. The method of claim 2, wherein the deletion is on chromosome 17p13.

5. The method of claim 1, wherein the gene identified as an independent prognostic factor specific for a disease is TP53, wherein said disease is cancer.

6. The method of claim 6, wherein the cancer is multiple myeloma.

7. A method for identifying a gene relevant in prognosis of a disease, comprising:
isolating plasma cells from individuals within a population;
extracting nucleic acid from said plasma cells;
hybridizing said nucleic acid to a DNA microarray; and
performing log rank test on the data obtained from said hybridization to identify genes that are up-regulated and down-regulated in the plasma, thereby identifying the gene important for prognosis of the disease.

8. The method of claim 7, further comprising:
analyzing nucleic acid obtained from the plasma cells; and
performing log rank test on data obtained after analyzing the nucleic acid, wherein said test correlates the status of the gene with progression and outcome of the disease.

9. The method of claim 8, wherein said analysis of the nucleic acid comprises determining mRNA expression of the gene, sequence integrity of the gene, copy number of the gene or a combination thereof.

10. The method of claim 1, further comprising:
performing gene expression profiling to identify genes associated with the gene linked to survival specific for the disease.

11. The method of claim 1, wherein said genes are selected from the group consisting of TRIM13, NADSYN1, TRIM22, AGRN, CENTD2, SESN1, TM7SF2, NICKAP1, COPG, STAT3, ALOX5, APP, ABCB9, GAA, CEP55, BRCA1, ANLN, PYGL, CCNE2, ASPM, SUV39H2, CDC25A, IFIT5, ANKRA2, PHLDB1, TUBA1A, CDCA7, CDCA2, HFE, RIF1, NEIL3, SLC4A7, FXYD5, MCC, MKNK2, KLHL24, DLC1, OPN3, B3GALNT1, SPRED1, ARHGAP25, RTN2, WNT16, DEPDC1, STT3B, ECHDC2, ENPP4, SAT2, SLAMF7, MAN1C1, INTS7, ZNF600, L3MBTL4, LAPTM4B, OSBPL10, KCNS3, THEX1. CYB5D2, UNC93B1, SIDT1, TMEM57, HIGD24, FKSG44, C14orf28, LOC387763, TncRNA, C18orf1, DCUN1D4, FANCI, ZMAT3, NOTCH1, BTG2, RAB1A, TNFRSF10B, HDLBP, RIT1, KIF2C, S100A4, MEIS1, SGOL2, CD302, C5orf34, FAM111B and C18orf54.

12. The method of claim 1, wherein said method correlates the expression of the gene to survival of an individual suffering from the disease, with molecular classification of the disease, with molecular risk stratification of the disease to predict outcome or a combination thereof.

13. The method of claim 12, wherein low expression of said gene correlates with the high-risk molecular classification of the disease.

14. The method of claim 13, wherein the high-risk molecular classification of multiple myeloma is characterized by increased combined expression of MMSET, MAF/MAFB and PROLIFERATION signatures.

15. The method of claim 7, wherein said prognosis comprises a shorter event-free and overall survival.

16. The method of claim 7, wherein the gene is TP53 and the disease is cancer.

17. The method of claim 16, wherein the cancer is multiple myeloma.

18. A method for determining prognosis of an individual with multiple myeloma, comprising:
obtaining plasma cells from the individual; and
determining expression of TP53 alone or in combination with one or more genes selected from the group consisting of TRIM13, NADSYN1, TRIM22, AGRN, CENTD2, SESN1, TM7SF2, NICKAP1, COPG, STAT3, ALOX5, APP, ABCB9, GAA, CEP55, BRCA1, ANLN, PYGL, CCNE2, ASPM, SUV39H2, CDC25A, IFIT5, ANKRA2, PHLDB1, TUBA1A, CDCA7, CDCA2, HFE, RIF1, NEIL3, SLC4A7, FXYD5, MCC, MKNK2, KLHL24, DLC1, OPN3, B3GALNT1, SPRED1, ARHGAP25, RTN2, WNT16, DEPDC1, STT3B, ECHDC2, ENPP4, SAT2, SLAMF7, MAN1C1, INTS7, ZNF600, L3MBTL4, LAPTM4B, OSBPL10, KCNS3, THEX1. CYB5D2, UNC93B1, SIDT1, TMEM57, HIGD24, FKSG44, C14orf28, LOC387763, TncRNA, C18orf1, DCUN1D4, FANCI, ZMAT3, NOTCH1, BTG2, RAB1A, TNFRSF10B, HDLBP, RIT1, KIF2C, S100A4, MEIS1, SGOL2, CD302, C5orf34, FAM111B and C18orf54; and
comparing the expression level of the gene(s) with expression level of the gene in a control individual such that genes that are up-regulated, down-regulated or a combination thereof compared to gene expression levels in plasma cell of a control individual indicates prognosis of said individual.

19. The method of claim 1, wherein the individual with poor prognosis has up-regulated expression of one or more genes selected from the group consisting of CEP55, BRCA1, ANLN, PYGL, CCNE2, ASPM, SUV39H2, CDC25A, TUBA1A, CDCA7, CDCA2, HFE, RIF1, NEIL3, SLC4A7, OPN3, B3GALNT1, SPRED1, DEPDC1, ENPP4, INTS7, L3MBTL4, THEX1, DCUN1D4, FANCI, ZMAT3, NOTCH1, BTG2, RAB1A, TNFRSF10B, HDLBP, RIT1, KIF2C, S100A4, MEIS1, SGOL2, CD302, C5orf34, FAM111B and C18orf54; and has down-regulated expression of TP53 alone or in combination with one or more genes selected from the group consisting of TRIM13, NADSYN1, TRIM22, AGRN, CENTD2, SESN1, TM7SF2, NICKAP1, COPG, STAT3, ALOX5, APP, ABCB9, GAA, IFIT5, ANKRA2, PHLDB1, FXYD5, MCC, MKNK2, KLHL24, DLC1, ARHGAP25, RTN2, WNT16, STT3B, ECHDC2, SAT2, SLAMF7, MAN1C1, ZNF600, LAPTM4B, OSBPL10, KCNS3, CYB5D2, UNC93B1, SIDT1, TMEM57, HIGD24, FKSG44, C14orf28, LOC387763, TncRNA and C18orf1.

20. The method of claim 1, wherein the poor prognosis comprises a shorter event-free and overall survival, a high-risk subtype of the multiple myeloma or both.

21. The method of claim 20, wherein the high-risk subtype of multiple myeloma is further characterized by increased combined expression of MMSET, MAF/MAFB and PROLIFERATION signatures.

22. The method of claim 18, wherein the gene expression is determined by RT-PCR or DNA microarray.

23. The method of claim 18, wherein said control individual is a normal healthy individual.

24. A kit for prognosis of multiple myeloma, comprising:
nucleic acid probes complementary to mRNA of genes described in claim 18; and
written instructions for extracting nucleic acid from plasma cells of an individual and hybridizing said nucleic acid to the DNA microarray.
